(19) United States
(12) Patent Application Publication
(10) Pub. No.: US 2009/0324705 A1
(43) Pub. Date: Dec. 31, 2009

(54) PHYTONUTRIENT COMPOSITIONS FOR TOPICAL USE

(76) Inventor: Nina VIKHRIEVA, Moscow (RU)

Correspondence Address: STEMEDICA CELL TECHNOLOGIES, INC, 5375 MIRA SORRENTO PLACE, SUITE 100, SAN DIEGO, CA, 92121, US

(21) Appl. No.: 12/369,703

(22) Filed: Feb. 11, 2009

Related U.S. Application Data
(60) Provisional application No. 61/028,446, filed on Feb. 13, 2008.

(51) Int. Cl. A61K 9/127; A61K 38/54; A61K 36/00; A61K 36/82; A61K 36/45; A61K 36/886; A61K 36/752; A61K 36/28; A61K 36/73; A61P 17/02

(52) U.S. Cl. 424/450; 424/94.3; 424/725; 424/729; 424/732; 424/744; 424/736; 424/764; 424/765; 977/907

(57) ABSTRACT

Topical anti-oxidant liposomal phytonutrient formulations and methods for their use such as the treatment of skin inflammation and skin-lightening applications.

This application claims priority from provisional application Ser. No. 61/028,446, filed Feb. 13, 2008, the entire contents of which are incorporated herein by reference.

### FIELD OF THE INVENTION

The invention is in the field of topical neutraceuticals. In particular, the invention relates to compositions comprising liposomes encapsulating at least one botanical constituent. The invention further relates to methods of making and using the composition in the treatment of a variety of skin conditions.

### BACKGROUND

The topical application of cosmetic and therapeutic botanical skin compositions are known in the art. Many of these botanical compositions impart their therapeutic benefits through the introduction of anti-oxidants in a therapeutically effective amount. Examples of phytonutrient compositions are available in the art including: U.S. Pat. No. 4,857,325 which describes antioxidant compositions prepared by the water extraction of natural antioxidants from plant substrates; U.S. Pat. No. 4,923,697 which, describes a water soluble antioxidant obtained from plants of the order Chenopodiales; U.S. Pat. No. 5,124,167 which describes cosmetic compositions including a cosmetically acceptable carrier and an effective amount of a water soluble antioxidants, derived from plant tissues, which is capable of being absorbed, into mammalian skin to reduce the peroxide level; and U.S. Pat. No. 5,204,105 which describes an emulsified cosmetic composition derived from the group consisting of plant extracts, yeast extracts, and a combination thereof.

Known skin care botanical compositions however suffer from limited absorption and lack efficacy in the treatment of skin conditions such as inflammation. Moreover, these compositions lack efficacy in applications such as the cosmetic lightening of the skin. What is needed in the art therefore is a naturally derived, antioxidant rich topical skin care composition that is effective in skin-lightening applications and the treatment of skin inflammation.

### SUMMARY OF THE INVENTION

An objective of the invention is to provide an antioxidant composition comprising liposomes in a pharmaceutical carrier suitable for topical application, wherein said liposomes comprise at least one botanical constituent selected from the group consisting of: barley grass juice powder, spirulina, chlorella, blueberry, green tea extract, grape seed extract, cranberry, raspberry, tart cherry, pine bark extract, broccoli, tomato, bilberry, elderberry, pomegranate, blackberry, isoquercitin/rutin (50/50), raspberry extract, apple, carrot, mango, sweet potato, lemon, parsley, peach, kale, broccoli, spinach, leek, beet, cranberry (quinic acid 6%), acerola cherry powder, rice bran, aloe vera powder extract, green sea, white tea, poygonum cuspidatum, oat beta glucan, cinnamon extract, cinnamon bark powder, milk thistle, marigold extract, dunaliella salina, alpha amylase, bromelain, cellulose, galactosidase, glucoamylase, hemicellulase, lipase, papain, lecithin, cabbage, lycopene extract, lemon peel powder, quinoa sprout, artichoke extract, and atlantic kelp powder.

A further objective of the invention is to provide an antioxidant composition comprising liposomes in a pharmaceutical carrier suitable for topical application, wherein said composition is capable of treating EMR-induced complications of the skin.

A further objective of the invention is to provide an antioxidant composition comprising liposomes in a pharmaceutical carrier suitable for topical application, wherein said composition is effective in the treatment of an inflammatory disorder selected from the group consisting of sunburn, non-ablative laser therapy, laser post-operative chemical peeling, dryness, post-operative trauma, post-operative damage of the mucosa, chemical/radioactive therapy of oncology patients, alopecia, burns, rejuvenation of hair follicles, insect stings and insect bites.

A further objective of the invention is to provide an anti-oxidant composition comprising liposomes in a pharmaceutical carrier suitable for topical application, wherein said composition is effective in lightening the skis.

### BRIEF DESCRIPTION OF THE DRAWINGS

FIGS. 1 and 2 depict human forearm skin after irradiation with a laser at varying intensities.

FIGS. 3 and 4 depict human forearm skin after irradiation with a laser at varying intensities, and after treatment with a phtyonutrient composition of the invention.

FIGS. 5 and 6 are photographs depicting a split face trial of a patient 24 hours after laser irradiation of the patient's entire face, wherein a phytonutrient composition of the invention was applied only to the left side of the patient's face.

FIG. 7 is a photographs depicting a split face trial of a patient 72 hours after laser irradiation of the patient's entire face, wherein a phytonutrient composition of the invention was applied only to the left side of the patient's face.

### DEFINITIONS

“Transcutaneous,” “transepithelial” and “transepidermah” are used interchangeably herein to refer to a substance, that when applied topically to the skin of a mammal, is capable of penetrating the skin. Transcutaneous substances may penetrate one or more of the stratum corneum, stratum granulosum, stratum spinosum, stratum basale (basal cell layer), dermis and subcutaneous tissue.

A “transferosome,” or “elastic vesicle,” is used to refer to a transcutaneous liposome that is capable of encapsulating, and delivering, one or more botanical constituents to one or more of the stratum corneum, stratum granulosum, stratum spinosum, stratum basale (basal cell layer), dermis and subcutaneous, tissue.

As used herein, the terms “treat,” “treating” and “treats” refer to the application of a therapeutic substance (i.e. the compositions of the invention) in the prevention, improvement or reversal of the symptoms of a disease or disorder.

The phrase “disease or condition” refers to any pathology that can be prevented, improved or reversed through the application of a phytonutrient composition of the inventive composition.

As used herein, the term “EMR” means electromagnetic radiation (EMR) ranging between, and including, 1 nanometer and 1 meter.

As used herein, the phrase “EMR-induced complications of the skin” refers to pathological changes in the skin due to exposure to EMR, including, but not limited to, those changes resulting from sun exposure, laser, and particle radiation.

As used herein, the phrase “phytonutrient composition” refers to a formulation that comprises at least one botanical liposome.

As used herein, the phrase “suitable for application to the skin,” or “suitable for topical application,” is used to describe a pharmaceutical carrier that is non-toxic and capable of placing botanical liposomes in contact with the skin under conditions that permit the botanical liposomes to penetrate at least one of the stratum corneum, stratum granulosum, stratum spinosum, stratum basale (basal cell layer), dermis and subcutaneous tissue.

As used herein, the terra “gel” refers to a composition that is of suitable viscosity for such purposes, e.g., a composition that is of a viscosity that enables it to be applied and remain on the skin.

As used herein, the phrase “botanical liposome” refers to a liposome that encapsulates a botanical constituent derived from plant matter.

As used herein, the phrase “an area of skin bearing an EMR-induced complication” refers to single or multiple skip sites or areas.

### INCORPORATION BY REFERENCE

All publications, including patents and patent applications, mentioned in this specification are incorporated by reference to the same extent as if each individual publication was set forth verbatim in the present disclosure.

### DETAILED DESCRIPTION

The inventor has discovered phytonutrient compositions, and methods for their use is the treatment of a variety of skin disorders, conditions and diseases. In particular, the inventor has discovered the usefulness of the phytonutrient compositions in treating inflammatory and damaging skin disorders.

### Botanical Constituents

The phytonutrient compositions of the invention are formulated using botanical liposomes. Botanical liposomes are liposomes that encapsulate one or more beneficial, plant-derived materials. Such materials may include any plant-derived substance that is useful in the treatment of at least one of the skin conditions disclosed herein. Beneficial plant-derived materials may contain one or more antioxidants, vitamins, minerals, or a combination thereof. In one aspect of the invention, the botanical liposomes of the invention encapsulate plant-derived materials that are rich in antioxidants.

The botanical liposomes of the invention may be formulated using a wide variety of plant-derived materials. These materials include, but are not limited to, barley grass, spirulina, chlorella, blueberry, green tea, grape seed, cranberry, coffee cherry, raspberry, tart cherry, pine bark, broccoli, tomato, bilberry, elderberry, pomegranate, blackberry, isoquercitin/rutin (50/50), raspberry, apple, carrot, mango, sweet, potato, lemon, parsley, peach, kale, broccoli, spinach, leek, beet, cranberry (quinic acid 6%), acerola, cherry, rice bran, aloe vera, green tea, white tea, poygonum cuspidatum, oat beta glucan, cinnamon extract, cinnamon bark, milk thistle, marigold, dunaliella salina, alpha amylase, bromelain, cellulose, galactosidase, glucoamylase, hemicellulase, lipase, papain, lecithin, cabbage, lycopene, lemon peel, quinoa sprout, artichoke, Atlantic kelp and combinations thereof. Although specific botanical constituents are called out in the present disclosure, one skilled in the art will appreciate that the methods and composition of the invention may be practiced, for example, using one or more of the botanical constituents discussed in the following references, the disclosures of which are incorporated herein by reference: U.S. Pat. Nos. 4,857,325, 4,923,697, 5,124,157, 5,204,105, 6,180,662, and 6,861,078; U.S. Pat. App. No. 2003/01385.07; and PCT App. No. 2006/068759.

In some aspects of the invention, the botanical constituents assume the form of an extract. Botanical extracts for use with the invention are concentrated preparations of the molecular constituents of a plant material. Such extracts may exist in a solid, gel or liquid form and may have varying levels of concentration. The concentration of the extracts may vary due to the presence of solvents; used in their preparation, or due to the use of a bulking agent (e.g. liquid or powdered materials that are added to increase the volume of the botanical constituent). One skilled in the art will appreciate the range of bulking agents suitable for use with the invention and may consult publications such as, for example. Remington's Pharmaceutical Sciences, Mack Publishing Go, (A. R. Gennaro edit 1985), the disclosure of which is incorporated herein by reference.

In some aspects of the invention, the botanical constituents may assume the form of a plant-derived isolate. That is, the invention's botanical liposomes may be formulated to encapsulate one or more purified, plant-derived molecules. Examples of isolates for encapsulation by the botanical liposomes include, but are not limited to, chlorogenic acid, caffeic acid, ferulic acid, quinic acid, trigonelline, proantocyanidins, gallic, epigallocatechin, trolox, merycetin, cyaniding, sterols, tocopherols, diterpenic alcohols, cafestol, kahweol and kauranic derivatives, and polyphenols. The botanical liposomes may encapsulate one or more of these isolates, or encapsulate these isolates in combination with another plant-derived preparation such as an extract.

One skilled in the art will appreciate that there are many methods which are suitable and adaptable for obtaining a desired plant-derived isolate. The selection of these methods will vary depending on the particular isolate that is desired. For example, US Pat. App. No. 20060263508, the disclosure of which is incorporated herein by reference, teaches the isolation of highly concentrated polyphenols (e.g., coffee acids, including caffeic, chlorogenic, ferulic acids) using basic ion exchange resin. Other methods for obtaining plant-derived isolates (e.g. polyphenols molecules) are taught by the following references, the disclosures of which are incorporated herein by reference: US Pat. App. Nos. 20050266104 and 20010021308; Chandra, A., et al., J. Agric. Food Chem. 41:1062 (1992); Wang, H., et al., J. Agric. Food Chem. 45:2556-2560 (1997); and Arora, A. and G. M. Strasburg, J. Amer. Oil Chem. Soc. 74:1031-1040 (1997).

### Botanical Liposomes

In general terms, the phytonutrient compositions of the invention may be practiced with any liposome, or combination of liposomes, that is capable of encapsulating at least one botanical constituent. Liposomes for use with the invention are non-toxic to living cells and may assume a unilamellar or multilamellar structure. Multilamellar liposomes have alternating hydrophobic and hydrophilic phases. Suitable nanosomes for formulating the composition of the invention and disclosed in U.S. Pat. Nos. 6,610,322 and 6,958,160, the disclosures of which are incorporated herein by reference.

In preferred embodiments, botanical liposomes for use with the invention are transferosomes which capable of permeating the skin to deliver an effective amount of plant-derived material. Such botanical transferosomes are sufficiently small in size, and elasticity, to permit them to penetrate the surface of the skin. Such botanical transferosomes come in a variety of sizes including, small liposomes, or nanosomes being, less than 25 nm, and intermediate-sized liposomes between about 25 nm and 500 nm. In some embodiments of the invention, the botanical transferosomes are between about 50 nm and 400 nm. The phytonutrient compositions of the invention may be formulated using botanical transferosomes of the same or different sizes. For example, the phytonutrient composition may be made with botanical transferosomes less than 25 nm in combination with intermediate-sized transferosomes of about 100 nm.

Botanical transferosomes must have sufficient elasticity to enable them to penetrate the surface of the skin. That is, such botanical transferosomes must be elastic enough to permit them to pass through at least one skin structure (e.g. the pores and/or stratum corneum), while remaining resistant to breakage. The elasticity required for the transferosomes of the invention is a function of their size as larger transferosomes require greater elasticity for proper permeation of the skin. For example, a 100 nm transferosome would have to have at least a five-fold deformation/elongation factor in order to penetrate the skin. Transferosomes with sufficient size and elasticity to penetrate the skin are known in the art. The following publications, which are incorporated by reference, provide the instructions necessary for manufacturing such botanical liposomes (i.e. transferosomes); Cevc et al. Adv Drug Deliv Reviews 56:675-711 (2004); and Cevc et al. Biochim Biophys Acta 1104:226-232 (1992); Cevc et al. J Control Release 36:3-16 (1995); Cevc et al. Biochim Biophys Acta 1368:201-215 (1998); Cevc et al. Clin Parmacokinet 42:461-474 (2003); and Elsayed et al. Pharmazie 62: 133-137 (2007).

The botanical liposomes of the invention may be derived from a wide variety of materials including, but not limited to, natural and synthetic phospholipids, glycolipids and other lipids and lipid congeners; cholesterol, cholesterol derivatives and other cholesterol congeners; charged species which impart a net charge to the membrane; reactive species which can react after liposome formation to link additional molecules to the liposome membrane; other lipid soluble compounds which have chemical or biological activities; and combinations thereof. More particularly, the liposomes of the invention may be made from polyethylene glycol [e.g. PEG-23 glycerol dipalmitate (PEG-23 GDP)], lecithin (e.g. soy lecithin), phosphatidylcholine (PC), sphingomyelin, cholesterol, phosphatidyiglycerol (PG) [e.g. fully saturated PG, such as egg PG (EPG) and diphosphatidylgiycerol], phosphatidylserine (PS), phosphatidylinositol (PI), lysophosphatide, phosphatide acid, phoshatidylethanolamine (PE), dimyristoyl phosphatidylcholine (DMPC), dimyristoyl phosphatidyiglycerol (DMPG), and combinations thereof. When formed from PC, the liposomes of the invention may be derived from unsaturated phosphatidylcholine (PC), egg PC (EPC), soy PC (SPC), fully or partially hydrogenated egg PC (HEPC), soy PC (HSPC) and combinations thereof. It is also, within, the scope of the invention to improve the stability of the liposomes using cholesterols (e.g. cholesterol esters, cholesterol hemisuccinate and cholesterol sulphates) as known in the art.

Encapsulation of the desired agent in liposomes may be effected by combining a phospholipid component with an aqueous component containing the desired botanical constituents under conditions which will result in vesicle formation. The phospholipid concentration must be sufficient to form lamellar structures, and the aqueous component must be compatible with the agent to be encapsulated. Methods for combining the phospholipid and the aqueous components so that vesicles will form include: drying the phospholipids onto glass and then dispersing them in the aqueous components; injecting phospholipids dissolved in a vaporizing or non-vaporizing organic solvent into the aqueous component which has previously been heated; and dissolving phospholipids in the aqueous base with detergents and then removing the detergent by dialysis. The liposomes can be produced from the foregoing mixtures either by sonication or by dispersing the mixture-through either small bore tubing or through the small orifice of a French Press. The methods for producing the liposomes as set forth in U.S. Pat. No. 5,077,211 to Yarosh are incorporated herein by reference. Suitable methods for liposome manufacture include, but are not limited to, thin film hydration, injection (e.g. crossflow injection, ethanol injection and ether injection), demulsification, hand shaking, spontaneous vesiculation, and liposomes from preformed vesicles (e.g. fusion, freeze-thawing, dehydration-rehydration and the Cochleate method).

It is within the scope of the present invention to use other methods for encapsulating botanical constituents within a liposome. A specific example of producing such botanical liposomes would include the following process. A lipid mixture as set forth above is dissolved in an organic solvent and dried to a thin film in a glass vessel. The selected botanical-constituents are purified and added to the vessel at high concentrations in an aqueous buffer to rehydrate the lipid. The mixture is then agitated by vortexing, and then sonicated to form liposomes. The liposome spheres containing the encapsulated botanical constituents) are then separated from the unincorporated agent by centrifugation or gel filtration.

Examples of liposomes suitable for use with the invention and their methods of manufacture include, but are not limited to, those taught in the following publications, the disclosures of which are incorporated by reference: Contreras et al. Int J. Pharm. 2005 Jun. 13; 297(1-2):134-45; Lee et al. Biol Pharm Bull. 2007 February; 30(2):393-6; Wen et al. Arch Pharm Res. 2006 December; 29(12):1187-92; Gong et al. PDA J Pharm Sci Technol. 2006 July-August; 60(4):259-63; Mishra et al. J Pharm Sci. 2007 January; 96(1):145-55; U.S. Pat. Nos. 3,957,971, 4,089,801, 4,196,191, 4,235,871, 4,485,054, 4,508,703, 4,731,210, 4,761,288, 4,804,539, 4,853,228, 4,897,269, 4,937,078, 4,963,297, 4,975,282, 5,008,050, 5,059,421, 5,169,637, 5,256,422, and 6,824,785.

In some aspects of the invention, the phytonutrient composition is formulated using a commercially available botanical liposome (i.e. transferosome) preparation. One non-limiting example of such a preparation is NanoGreens 10® (BioPharma Scientific, Inc., San Diego, Calif., USA).

Although the present disclosure details the liposomal encapsulation of botanical constituents, one skilled in the art will appreciate that invention may be practiced using a composition that encapsulates botanical constituents by other means, including, but not limited to, maltodextrin capsules, silica gels, and siloxanes. These encapsulation methods may be used in alone, in combination with one another, or in combination with botanical liposomes. Suitable methods for encapsulation of botanical constituents are disclosed in the following references, the disclosures of which are incorporated by reference; U.S. Pat. Nos. 6,825,161; 6,238,650; 6,436,375, 6,303,549; 6,468,509 6,436,375; 6,238,650; 6,468,509, 6,362,146; 6,074,630; 5,455,048; 5,770,556; 5,955,409; 5,876,755; 4,803,195; 5,508,259; 4,749,501; 6,248,703; 5,476,660; and 4,904,524 and EP Pat. Nos. 0,254,447; 0,025,379; and 0,399,911.

### Formulating the Compositions of the Invention

The phytonutrient compositions of the invention may be practiced using any formulation that is non-toxic to living cells and does not inhibit the therapeutic activity of the composition's botanical liposomes.

In some embodiments of the invention, the phytonutrient compositions are formulated such that they are suitable for topical application to the skin. Such topical formulations are preferably of a sufficient viscosity to permit the phytonutrient composition's botanical liposomes to remain on, and penetrate one or more structures of the skin (e.g. pores, hair follicles, stratum corneum, stratum granulosum, stratum, spinosum, stratum basale (basal cell layer), dermis and subcutaneous tissue). Topical formulations suitable for use the invention may assume die form of a gel, liquid, powder, paste (e.g. stick), cream (i.e. lotion), wax or foam. One skilled in the art will appreciate the formulations necessary for achieving phytonutrient composition of suitable viscosity for application to the skin.

In some embodiments of the invention, the phytonutrient compositions are formulated using a pharmaceutically acceptable carrier. As used herein the phrase “pharmaceutically acceptable carrier” means a suitable vehicle including one or more solid, semisolid or liquid diluents, excipients or encapsulating substances which are suitable for administration to the skin. Such carriers may be used to adjust the concentration of botanical liposomes and/or the viscosity of the phytonutrient compositions. Suitable carriers for use with the invention are non-toxic and do not interfere with the therapeutic effects of the botanical liposomes disclosed herein. Any suitable pharmaceutically acceptable carrier may be used with the invention, as will be readily apparent to one of ordinary skill in the art. Pharmaceutically acceptable carriers include, but are not limited to, hydroxypropyl cellulose, starch (corn, potato, rice, wheat), pregelatinized starch, gelatin, sucrose, acacia, alginic acid, sodium alginate, guar gum, ethyl cellulose, carboxymethylcellulose sodium, carboxymethylcellulose calcium, polyvinylpyrrolidone, methylcellulose, hydroxyproply methylcellulose, microcrystalline cellulose, polyethylene glycol, powdered cellulose, glucose, croscarmellose sodium, crospovidone, polacrilin potassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, castor oil, mineral oil, light mineral oil, glycerin, sorbitol, mannitol, stearic acid, sodium lauryl sulfate, hydrogenated vegetable oil (e.g., peanut, cottonseed, sunflower, sesame, olive, corn, soybean), zinc stearate, ethyl oleate, ethyl laurate, agar, calcium silicate, magnesium silicate, silicon dioxide, colloidal silicon dioxide, calcium chloride, calcium sulfate, silica gel, castor oil, diethyl phthalate, glyercin, mono- and di-acetylated monoglycerides, propylene, glycol, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, polyvinyl alcohol, povidine, sodium alginate, tragacanth, xanthan gum, and silicones.

The phytonutrient compositions of the invention may be formulated using any concentration of botanical liposomes that provides a therapeutic effect when the compositions are used according to the methods disclosed herein. One skilled in the art will appreciate the concentration of botanical liposomes that a desired outcome will require. In a preferred embodiment, the phytonutrient compositions comprise a botanical liposome concentration of about 0.5-5%. Some non-limiting examples of other suitable botanical liposome concentrations include about 0.5-0.7%, about 0.5-1.0% and about 1.0-1.5%.

In some aspects of the invention, topical phytonutrient compositions are formulated with additives that benefit the skin (i.e. skin actives). Suitable skin actives for use with the phytonutrient compositions include, but are not limited to, sunless tanning actives, skin lightening actives, anti-acne actives, anti-skin wrinkling and anti-skin aging actives, vitamins, anti-inflammatory actives, anesthetic actives, analgesic actives, anti-pruritic actives, anti-microbial actives (e.g. antifungals, antibacterials, and antiparasitics), anti-virals, anti-allergenics, medicinal actives (e.g., skin rash, skin disease and dermatitis-medications), anti-cellulite additives, insect repellant actives, antioxidants, hair growth promoters, hair growth inhibitors, hair bleaching agents, deodorant compounds, and mixtures and combinations thereof. Suitable skis actives for use with the invention are also disclosed in U.S. Pat. No. 7,025,952, the disclosure of which is incorporated herein by reference

Depending on the particular application that, a phytonutrient composition is intended for, the phytonutrient composition may assume the form of a consumer product such as, for example, liquid and bar soap (e.g. body wash); a shampoo; a hair conditioner; a shower gel; including an exfoliating shower gel; a foaming bath product (e.g. gel, soap or lotion); a milk bath; a soapless cleanser, including a gel cleanser, a liquid cleanser and a cleansing bar; moist towelletes; a body lotion; a body spray, mist or gel; bath effervescent tablets (e.g., bubble bath); a hand and nail cream; a bath/shower gel; a shower cream; a depilatory cream; a shaving product e.g. a shaving cream, gel, foam or soap, an after-shave, after-shave moisturizer; or sunscreen.

Some non-limiting examples of phytonutrient compositions for use with the invention are presented in Tables 1-6 below.

### Methods of Use

The compositions of the invention find use in the treatment of any disease or condition, that benefits from the administration (e.g. topical administration) of one or more botanical constituents. One skilled in the art will appreciate that a selected administration route for the compositions of the invention may vary depending upon the condition that is being treated and the formulation that is being used.

The phytonutrient compositions of the invention may be used to treat a variety of skin conditions, including, but not limited to, inflammation of the skin (i.e. cutaneous inflammation) resulting from physical forces such as EMR, thermal burns, abrasions (e.g. cosmetic microderm abrasion) and the like. In the case of EMR-induced inflammation, the compositions of the invention can be used to treat burns resulting from radioactive therapy, lasering of the skin (e.g. after laser skin resurfacing, cosmetic laser-based wrinkle treatment, laser hair removal, laser depigmentation, laser tattoo removal, or non-ablative laser rejuvenation of the skin) and sun exposure (e.g. sunburn).

In some aspects of the invention, the phytonutrient compositions are used to treat inflammation of the skin resulting from eczema and related conditions, erythroderma, mycosis fungoides and related conditions, pyoderma gangrenosum, erythema multiforme, rosacea, onychomycosis, acne and related conditions, psoriasis, hyperkeratosis, allergic reactions (rashes, hives, burns), psoriasis, hemorrhoids, exposure to poisonous weeds (e.g. poison, oak, poison ivy and stinging nettle), insect stings and bites, jellyfish stings, microbial infection, such as by viral, fungal or bacterial pathogens. In some aspects of the invention, the compositions are used to treat fungal infections including, but not limited to, ringworm, jock-itch, diaper rash, and athletes foot.

In some aspects of the invention, the phytonutrient compositions are used to cosmetically rejuvenate the skin, such as on the face and hands. Such applications include, but are not limited to, moisturizing the skin (reducing dryness and cracking), reducing die appearance of lines and wrinkles, increasing firmness. The compositions of the invention also find use in the treatment of scalp conditions, such as, for example, dandruff and alopecia.

In some aspects of the invention, the phytonutrient compositions find use in skin lightening-applications. The compositions may be used to lighten specific sites on the skin (e.g. a hyperpigmented lesion), or large portions of the skin, such as, for example, whitening the skin of the face and neck. As used herein, “skin lightening” means decreasing melanin in skin, including one or more of, overall lightening of basal skin tone, lightening of hyperpigmented lesions, including age spots, melasma, chloasma, freckles, post inflammatory hyperpigmentation, port wine stain, or sun-induced pigmented blemishes.

In aspects of the invention, the disclosed skin conditions are treated through the topical application of the disclosed phytonutrient compositions. In such applications the phytonutrient compositions may applied to the skin in the form of a gel, liquid, powder, spray, mists, aerosol, ointment, emulsion, suspension, paste (e.g. stick), cream (i.e. lotion), wax or foam. The phytonutrient compositions may be applied to the skin through the use of a vehicle such as a patch or compress. One skilled in the art will appreciate the type of delivery method a particular skin condition will require.

TABLE 1
NanoGreen eye care repairing night cream

| | | | State standard, |
|---|---|---|---|
| N° | Components | % | component producer |
| 1 | Water | up to | [symbol] 2874-82 |
| | | 100 | |
| 2 | Cetearyl alcohol | 1.0-1.5 | “Vopelius Chemie AG”, |
| | | | Germany |
| 3 | Glyceryl stearate, ceteareth-20, | 2.0-3.0 | “Henkel”, Germany |
| | ceteareth-14, cetearyl alkohol, | | |
| | cetyl palmitate (Emulgade^{R}) | | |
| 4 | Glycerin | 3.0-4.0 | “Vopelius Chemie AG”, |
| | | | Germany |
| 5 | Isopropyl palmitate | 2.0-3.0 | “Vopelius Chemie AG”, |
| | | | Germany |
| 6 | Dimethicone | 1.0-2.0 | “Vopelius Chemie AG”, |
| | | | Germany |
| 7 | Propylene glycol | 2.0-3.0 | “Vopelius Chemie AG”, |
| | | | Germany |
| 8 | Ceteareth-6, cetearyl | 0.5-1.0 | “Vopelius Chemie AG”, |
| | alkohol (Cremofore A 6) | | Germany |
| 9 | Lenseed oil | 0.5-1.0 | “Vopelius Chemie AG”, |
| | | | Germany |
| 10 | Vitamin E | 0.5-1.0 | “Basf”, Germany |
| 11 | Nano Greens | 0.5-0.7 | “Biopharma”, USA |
| 12 | PEG-40 hydrogenated castor oil | 0.3-0.5 | “Vopelius Chemie AG”, |
| | | | Germany |
| 13 | Carbopol | 0.3-0.5 | “Vopelius Chemie AG”, |
| | | | Germany |
| 14 | Triethanolamine | 0.3-0.5 | TY 6-02-982-98 |
| 15 | Colza oil monoethanolamide | 0.3-0.4 | “Vopelius Chemie AG”, |
| | | | Germany |
| 16 | Phenoxyethanol | 0.3-0.5 | “Vopelius Chemie AG”, |
| | | | Germany |
| 17 | Phenonyp | 0.3-0.4 | “Vopelius Chemie AG”, |
| | | | Germany |
| 18 | Parfume | 0.3-0.4 | France |

TABLE 2
NanoGreen anti-age mask

| | | | State standart, |
|---|---|---|---|
| N° | Components | % | component producer |
| 1 | Water | up to | [symbol] 2874-82 |
| | | 100 | |
| 2 | Cetearyl alcohol | 3.0-4.0 | “Vopelius Chemie AG”, |
| | | | Germany |
| | Glyceryl stearat, cetearet-20, | 3.0-4.0 | “Henkel”, Germany |
| | cetearet-12, cetearyl alcohol, | | |
| | cetyl palmitat (Emulgade) | | |
| 4 | Glycerol | 4.0-5.0 | “Vopelius Chemie AG”, |
| | | | Germany |
| 5 | Isopropylpalmitate | 2.0-3.0 | “Vopelius Chemie AG”, |
| | | | Germany |
| 6 | Cocos oil | 2.0-3.0 | “Vopelius Chemie AG”, |
| | | | Germany |
| 7 | Parfume oil | 4.0-5.0 | “Vopelius Chemie AG”, |
| | | | Germany |
| 8 | Dimeticon | 2.0-3.0 | “Vopelius Chemie AG”, |
| | | | Germany |
| 9 | Propyleneglycol | 2.0-3.0 | “Vopelius Chemie AG”, |
| | | | Germany |
| 10 | Cetearet-6 and cetearyl | 0.5-1.0 | “Vopelius Chemie AG”, |
| | alcohol (creamfor A6) | | Germany |
| 11 | Wax emulgated | 1.0-2.0 | “Vopelius Chemie AG”, |
| | | | Germany |
| 12 | Parrafine | 0.5-1.0 | “Vopelius Chemie AG”, |
| | | | Germany |
| 13 | Lanoline anhydrous | 0.5-1.0 | “Vopelius Chemie AG”, |
| | | | Germany |
| 14 | Nano Greens | 1.0-1.5 | “Biopharma”, USA |
| 15 | Cocos oil | 1.0-2.0 | “Vopelius Chemie AG”, |
| | | | Germany |
| 16 | Titaniun dioxide | 1.0-2.0 | “Basf”, Germany |
| 17 | Flaxseed oil | 0.5-1.0 | “Vopelius Chemie AG”, |
| | | | Germany |
| 18 | Cyclometicon | 0.5-1.0 | “Vopelius Chemie AG”, |
| | | | Germany |
| 19 | Vitamin E | 0.3-0.5 | “Basf”, Germany |
| 20 | Caoline | 0.3-0.5 | TY 5729-071-00284530-96 |
| 21 | PEG-40 oleum ricini | 0.3-0.5 | “Vopelius Chemie AG”, |
| | hydrogenased | | Germany |
| 22 | Cocamidpropyl betain | 0.3-0.5 | “Vopelius Chemie AG”, |
| | | | Germany |
| 23 | Polyvinilpyrrolidon | 0.1-0.2 | “Basf”, Germany |
| 24 | Phenoxyethanol | 0.3-0.5 | “Vopelius Chemie AG”, |
| | | | Germany |
| 25 | Phenothype | 0.3-0.4 | “Vopelius Chemie AG”, |
| | | | Germany |
| 28 | Parfume composition | 0.3-0.4 | France |

TABLE 3
NanoGreen day cream for oily and combined skin

| | | | State standard, |
|---|---|---|---|
| N° | Component | % | component producer |
| 1 | Water | up to | [symbol] 2874-82 |
| | | 100 | |
| 2 | Cetearyl alcohol | 2.0-3.0 | “Vopelius Chemie AG”, |
| | | | Germany |
| 3 | Glyceryl stearat, cetearet-20, | 3.0-4.0 | “Henkel”, Germany |
| | cetearet-12, cetearyl alcohol, | | |
| | cetyl palmitat (Emulgade) | | |
| 4 | Glycerol | 3.0-4.0 | “Vopelius Chemie AG”, |
| | | | Germany |
| 5 | Isopropylpalmitate | 2.0-3.0 | “Vopelius Chemie AG”, |
| | | | Germany |
| 6 | Parfume oil | 2.0-3.0 | “Vopelius Chemie AG”, |
| | | | Germany |
| 7 | Dimeticon | 1.0-2.0 | “Vopelius Chemie AG”, |
| | | | Germany |
| 8 | Propyleneglycol | 2.0-3.0 | “Vopelius Chemie AG”, |
| | | | Germany |
| 9 | Cetearet-6 and cetearyl | 1.0-2.0 | “Vopelius Chemie AG”, |
| | alcohol (cremofor A6) | | Germany |
| 10 | Octylmethoxicynamat | 1.0-2.0 | “Basf”, Germany |
| 11 | Titanium dioxide | 1.0-1.5 | “Basf”, Germany |
| 12 | Nano Greens | 0.5-1.0 | “Biopharma”, USA |
| 13 | Vitamin E | 0.3-0.5 | “Basf”, Germany |
| 14 | [symbol] [symbol] | 0.5-1.0 | “Vopelius Chemie AG”, |
| | | | Germany |
| 15 | PEG-40 oleum ricini | 0.3-0.5 | “Vopelius Chemie AG”, |
| | hydrogenased | | Germany |
| 16 | Colza oil monoethanolamide | 0.3-0.5 | “Vopelius Chemie AG”, |
| | | | Germany |
| 17 | Sodium acrylate copolymer, | 0.2-0.3 | “Cognis”, Germany |
| | tridecent-6, parfume oil | | |
| 18 | PEG-7 glyceril cocoate | 0.3-0.5 | “Vopelius Chemie AG”, |
| | | | Germany |
| 19 | Polyvinilpyrrolidon | 0.1-0.2 | “Basf”, Germany |
| 20 | Carbopol | 0.2-0.3 | “Vopelius Chemie AG”, |
| | | | Germany |
| 21 | Parfume composition | 0.2-0.3 | TY 6-02-982-96 |
| 22 | Phenoxyethanol | 0.3-0.4 | “Vopelius Chemie AG”, |
| | | | Germany |
| 23 | Phenothype | 0.2-0.3 | “Vopelius Chemie AG”, |
| | | | Germany |
| 24 | Parfume composition | 0.3-0.4 | France |

TABLE 4
NanoGreen repairing night cream

| | | | State standard, |
|---|---|---|---|
| N° | Component | % | component producer |
| 1 | Water | up to | [symbol] 2874-82 |
| | | 100 | |
| 2 | Cetearyl alcohol | 2.0-3.0 | “Vopelius Chemie AG”, |
| | | | Germany |
| 3 | [symbol] | 3.0-4.0 | “Henkel”, Germany |
| | [symbol] | | |
| 4 | Glycerol | 3.0-4.0 | “Vopelius Chemie AG”, |
| | | | Germany |
| 5 | Isopropylpalmitate | 2.0-3.0 | “Vopelius Chemie AG”, |
| | | | Germany |
| 6 | Cocos oil | 3.0-4.0 | “Vopelius Chemie AG”, |
| | | | Germany |
| 7 | Parfume oil | 2.0-3.0 | “Vopelius Chemie AG”, |
| | | | Germany |
| 8 | Dimeticon | 1.0-2.0 | “Vopelius Chemie AG”, |
| | | | Germany |
| 9 | Propyleneglycol | 2.0-3.0 | “Vopelius Chemie AG”, |
| | | | Germany |
| 10 | Soya oil | 1.0-1.5 | “Vopelius Chemie AG”, |
| | | | Germany |
| 11 | Capryl caprylic | 0.5-1.0 | “Vopelius Chemie AG”, |
| | triglyceralde | | Germany |
| 12 | Nano Greens ™ | 0.5-1.0 | “Biopharma”, USA |
| 13 | Soya oil | 0.5-1.0 | “Vopelius Chemie AG”, |
| | | | Germany |
| 14 | Flaxseed oil | 0.5-1.0 | “Vopelius Chemie AG”, |
| | | | Germany |
| 15 | Cyclometicon | 0.5-1.0 | “Vopelius Chemie AG”, |
| | | | Germany |
| 16 | Vitamin E | 0.3-0.5 | “Basf”, Germany |
| 17 | Lanolin anhydrous | 0.5-1.0 | “Vopelius Chemie AG”, |
| | | | Germany |
| 18 | PEG-40 oleum ricini | 0.3-0.5 | “Vopelius Chemie AG”, |
| | hydrogenased | | Germany |
| 19 | Sodium acrylate | 0.2-0.3 | “Cognis”, Germany |
| | copolymer, tridecent-6, | | |
| | parfume oil | | |
| 20 | Phenoxiethanol | 0.3-0.5 | “Vopelius Chemie AG”, |
| | | | Germany |
| 21 | Phenothype | 0.3-0.4 | “Vopelius Chemie AG”, |
| | | | Germany |
| 22 | Parfume | 0.3-0.4 | France |

TABLE 5
NanoGreen anti-age day cream for dry and normal skin

| | | | State standard, |
|---|---|---|---|
| N° | Components | % | component producer |
| 1 | Water | up to | [symbol] 2874-82 |
| | | 100 | |
| 2 | Cetearyl alcohol | 2.0-3.0 | “Vopelius Chemie AG”, |
| | | | Germany |
| 3 | Glyceryl stearat, cetearet-20, | 3.0-4.0 | “Henkel”, Germany |
| | cetearet-12, cetearyl alcohol, | | |
| | cetyl palmitat (Emulgade) | | |
| 4 | Glycerol | 3.0-4.0 | “Vopelius Chemie AG”, |
| | | | Germany |
| 5 | Isopropylpalmitate | 2.0-3.0 | “Vopelius Chemie AG”, |
| | | | Germany |
| 6 | Cocos oil | 4.0-5.0 | “Vopelius Chemie AG”, |
| | | | Germany |
| 7 | Parfume oil | 2.0-3.0 | “Vopelius Chemie AG”, |
| | | | Germany |
| 8 | Dimeticon | 1.0-2.0 | “Vopelius Chemie AG”, |
| | | | Germany |
| 9 | Cetearet-6 and cetearyl | 1.0-2.0 | “Vopelius Chemie AG”, |
| | alcohol (creamfor A6) | | Germany |
| 10 | Soya oil | 0.5-1.0 | “Vopelius Chemie AG”, |
| | | | Germany |
| 11 | Flaxseed oil | 0.5-1.0 | “Vopelius Chemie AG”, |
| | | | Germany |
| 12 | Octylmethoxicynamat | 1.0-2.0 | “Basf”, Germany |
| 13 | Cyclometicon | 0.5-1.0 | “Vopelius Chemie AG”, |
| | | | Germany |
| 14 | Titaniun dioxide | 1.0-1.5 | “Basf”, Germany |
| 15 | Nano Greens | 0.5-1.0 | “Biopharma”, USA |
| 16 | Vitamin E | 0.3-0.5 | “Basf”, Germany |
| 17 | PEG-40 oleum ricini | 0.5-0.7 | “Vopelius Chemie AG”, |
| | hydrogenased | | Germany |
| 18 | PEG-7 glyceril cocoate | 0.3-0.5 | “Vopelius Chemie AG”, |
| | | | Germany |
| 19 | Phenoxyethanol | 0.3-0.5 | “Vopelius Chemie AG”, |
| | | | Germany |
| 20 | Polyvinilpyrrolidon | 0.1-0.2 | “Basf”, Germany |
| 21 | Phenotype | 0.3-0.4 | “Vopelius Chemie AG”, |
| | | | Germany |
| 22 | Parfume composition | 0.3-0.4 | France |

TABLE 6
NanoGreen Refreshing tonic

| | | | State standard, |
|---|---|---|---|
| N° | Components | % | component producer |
| 1 | Water | up to | [symbol] 2874-82 |
| | | 100 | |
| 2 | Propyleneglycol | 3.0-4.0 | “Vopelius Chemie AG”, |
| | | | Germany |
| 3 | Glycerol | 2.0-3.0 | “Vopelius Chemie AG”, |
| | | | Germany |
| 4 | Sodium lactate | 1.0-1.5 | “Purac”, Netherlands |
| 5 | Urea | 0.5-1.0 | “Vopelius Chemie AG”, |
| | | | Germany |
| 6 | PEG-7 glyceril cocoate | 0.5-0.7 | “Vopelius Chemie AG”, |
| | | | Germany |
| 7 | PEG-40 oleum ricini | 0.5-0.7 | “Vopelius Chemie AG”, |
| | hydrogenased | | Germany |
| 8 | Cocamidopropyl | 0.5-0.7 | “Vopelius Chemie AG”, |
| | | | Germany |
| 9 | Glicine | 0.1-0.2 | China |
| 10 | Euxil K-100 | 0.07-0.1 | “Vopelius Chemie AG”, |
| | | | Germany |
| 11 | Parfume composition | 0.3-0.4 | France |
| 12 | Tartrasine stain (CI 19140) | 0.002 | France |
| 13 | Blue stain (CI 19140) | 0.001 | France |

### Example 1

### Topical Phytonutrient Composition

A phytonutrient composition was formulated using NanoGreens 10® (BioPharma Scientific, Inc., San Diego, Calif., USA) as the source of the botanical liposomes. The composition was formulated as a gel according to well known principles in the art (see e.g. Handbook Of Pharmaceutical Excipients 2003, American Pharmaceutical Association, Washington, D.C., USA and Pharmaceutical Press, London, UK).

### Example 2

### Treatment of Inflammation in Human Skin

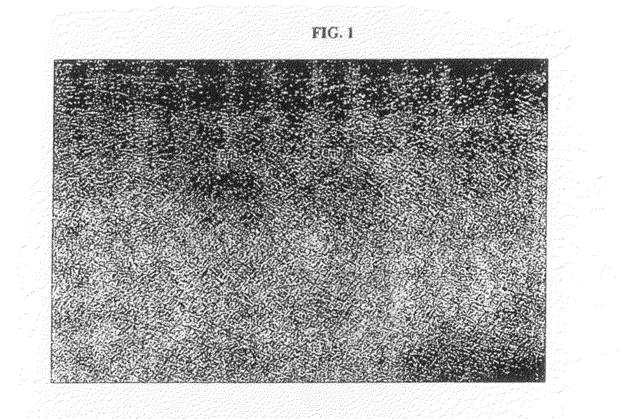

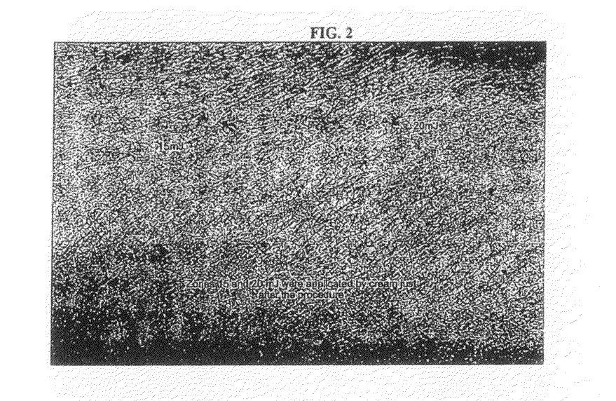

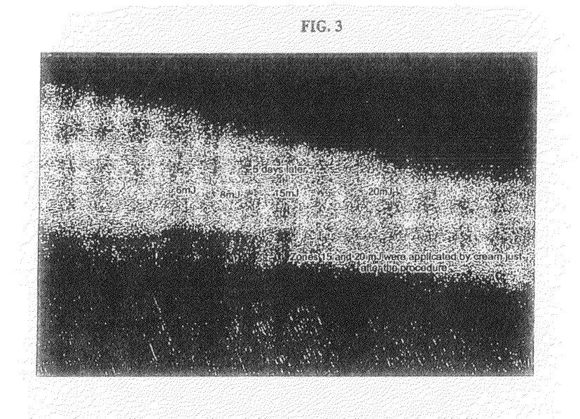

The forearm skin of a female was irradiated with a diode-pumped fiber laser (Fraxel™ Model 1500) with a 1.5 micrometer wavelength at energy levels of 6 mJ, 8 mJ, 15 mJ and 20 mJ. FIGS. 1-4 demonstrate that inflammation of the skin occurred at all laser intensities.

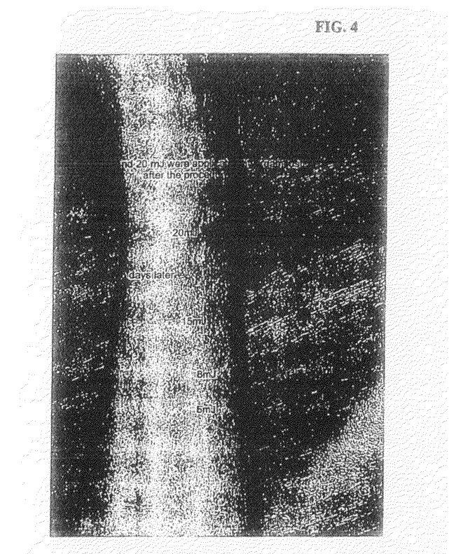

Human forearm skirt was irradiated at 4 adjacent locations at four different intensities: pulse energies of 6 mJ, 8 mJ, 15 mJ and 20 mJ, respectively (FIG. 4). Pulse durations were on the order of 1-2 milliseconds, the number of pulses was 500-2000/cm², and the spot diameter was 50-300 microns. Immediately following irradiation, the phytonutrient composition of Example 1 was applied to the regions irradiated at 15 mJ and 20 mJ. Comparison of these treated areas, with the untreated areas (e.g. areas irradiated at 6 mJ and 8 mJ) showed that the phytonutrient composition decreased the laser-induced inflammation of the skin, including reduction of skin erythema and edema.

### Example 3

### Treatment of Inflammation in Facial Skin

The facial skin of a male was irradiated with a 10.6 micron CO₂ laser (Fraxel®) under the following conditions: Immediately following, laser irradiation, the composition of Example 1 was applied only to the left side of the face. The pulse duration was less than 1 second. The energy was 2-25 mJ at a repetition rate from 1 Hz to 1 k Hz. Spot site was 50 microns to 3 mm.

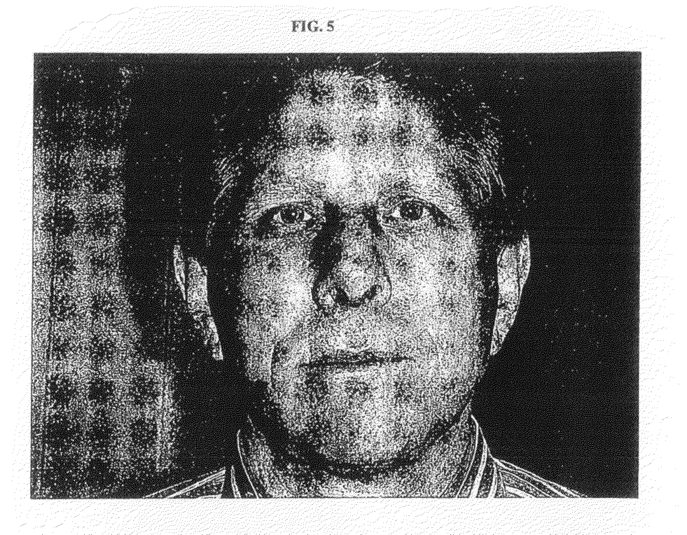

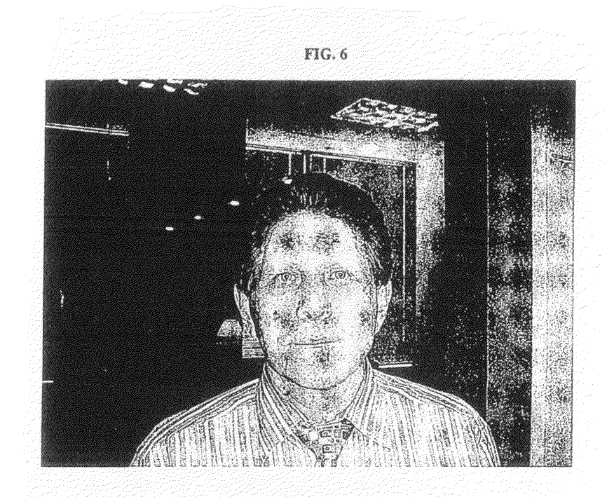

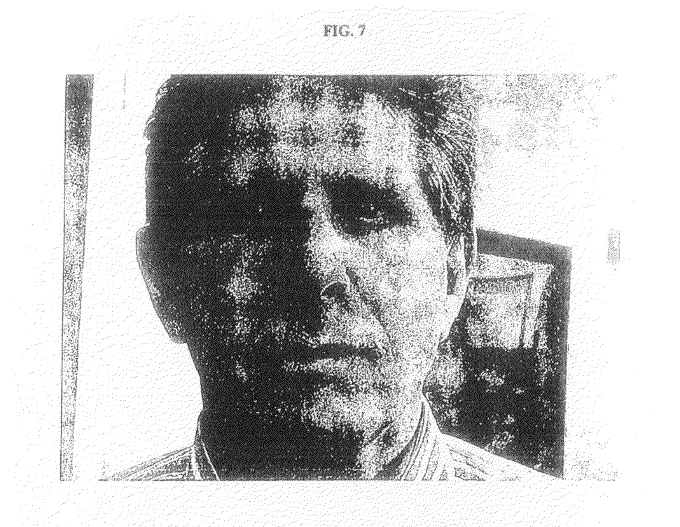

As shown in FIGS. 5 and 6, twenty four hours after laser treatment, the left side appeared as normal, caucasian skin, pale and not swollen. The right side was significantly swollen (edema), with redness (pronounced erythema). Twenty four hours later, the composition was applied to the right side. 48 hours later, the fight side appeared similar to the left (FIG. 7).

In other patients receiving the same laser treatment; the composition was not applied. For five to 7 days after laser treatment, the left and right sides appeared swollen and red.

These results show that the compositions of the invention and the methods of applying the compositions of the invention to inflammatory skin disorders are effective in significantly reducing inflammatory signs (e.g. erythema, edema, pain).

### Example 4

### Sunburn Study

Patients who incurred significant sunburn, with accompanying erythema and edema, within 24 hours of subathing were treated with the compositions and methods of the invention within 24 hours of the appearance of the sunburn. The sunburn symptoms were resolved within 24 hours after the treatment.

In another study, patients, prior to sunbathing, were treated with the compositions of the invention according to the methods of the invention. Compared to untreated patients, the signs (erythema, edema) of sunburn in the treated patients were reduced or prevented. It should be understood that application of the compositions to the skin should, be done not more than 1 to 2 hours prior to sunbathing or other light exposure.

In studies of treating human skin for a variety of light complications using the compositions of the invention and methods of using the compositions, similar successful results have been achieved by treating skin with any of the following lasers:
Laser with 810 nanometer wavelength, fluence of 0.2-50 J/cm²
1064 Laser with fluence of 30-200 J/cm²
532 Laser with fluence of 6-25 J/cm²
1.5 micrometer laser, fluence of 1-500/cm²

### Example 5

### Skin Lightening

Human facial skin is treated with the NanoGreens¹⁰ formulation of Example 1. The experiment is conducted as a split skin study wherein half of each subject's face is treated with the formulation, while the other half is untreated and maintained as a negative control. Treated areas will include sunspots of varying shades. The formulation is applied one or two times a day for seven to ten days. Facial skin treated with the NanoGreens¹⁰ formulation will appear several shades lighter than the untreated, negative control. The appearance of sun spots will also be decreased in the treated areas.

The results of the experiment will show that the phytonutrient composition of Example 1 is effective in the lightening the skin.

I claim:

1. A phytonutrient composition comprising liposomes in a pharmaceutical carrier, wherein said composition is suitable for topical application, and wherein said liposomes comprise at least one botanical constituent selected from the group consisting of:
barley grass juice powder, spirulina, chlorella, blueberry, green tea extract, grape seed extract, cranberry, raspberry, tart cherry, pine bark extract, broccoli, tomato, bilberry, elderberry, pomegranate, blackberry, isoquercitin/rutin (50/50), raspberry extract, apple, carrot, mango, sweet potato, lemon, parsley, peach, kale, broccoli, spinach, leek, beet, cranberry (quinic-acid 6%), acerola cherry powder, rice bran, aloe vera powder extract, green tea, white tea, poygonum cuspidatum, oar beta glucan, cinnamon extract, cinnamon bark powder, milk thistle, marigold extract, dunaliella salina, alpha amylase, bromelain, cellulose, galactosidase, glucoamylase, hemicellulase, lipase, papain, lecithin, cabbage, lycopene extract, lemon peel powder, quinoa sprout, artichoke extract, and atlantic kelp powder.

2. The composition of claim 1, wherein said composition is capable of treating an EMR-induced complication of the skin.

3. The composition of claim 2, wherein said complication is selected from the group consisting of inflammation, edema, erythema and perforation of epidermis and dermis.

4. The composition of claim 3, wherein said inflammatory disorder is selected from the group consisting of sunburn, non-ablative laser therapy, laser post-operative chemical peeling, dryness, post-operative trauma, post-operative damage of the mucosa, chemical therapy of oncology patients, radioactive therapy of oncology patients, alopecia, burns, rejuvenation of hair-follicles, insect stings and insect bites.

5. The composition of claim 1, wherein said composition comprises about 5% of said liposomes by weight.

6. The composition of claim 1, wherein said pharmaceutical carrier is selected from the group consisting of a gel, a paste, foam and a cream.

7. The composition of claim 1, wherein said pharmaceutical carrier is selected from the group consisting of aqueous, lipid, alcohol and combinations thereof.

8. The composition of claim 1, wherein said liposomes are of a size between about 0.1 and 200 nanometers.

9. An phytonutrient composition comprising nanosomes and a pharmaceutical carrier suitable for topical application, wherein said liposomes comprise barley grass juice powder, spirulina, chlorella, blueberry, green tea extract, grape seed extract, cranberry, raspberry, tail cherry, pine bark extract, broccoli, tomato, bilberry, elderberry, pomegranate, blackberry, isoquercitin/rutin (50/50), raspberry extract, apple, carrot, mango, sweet potato, lemon, parsley, peach, kale, broccoli, spinach, leek, beet, cranberry (quinic acid 6%), acerola cherry powder, rice bran, aloe vera powder extract, green tea, white tea, poygonum cuspidatum, oat beta glucan, cinnamon extract, cinnamon bark powder, milk thistle, marigold extract, dunaliella salina, alpha amylase, bromelain, cellulose, galactosidase, glucoamylase, hemicellulase, lipase, papain, lecithin, cabbage, lycopene extract, lemon peel powder, quinoa sprout, artichoke extract and atlantic kelp powder.

10. A method for treating an area of skin bearing an EMR-induced complication, said method comprising the step of applying an effective amount of the phytonutrient composition, of claim 1 to said area of skin.

11. A method for preventing an EMR-induced complication in an area of skin, said method comprising the step of applying an effective amount of the anti-oxidant composition of claim 1 to said area of skin prior to said skin being subjected to EMR.

12. A method for reducing an EMR-induced complication in an area of skin, said method comprising the step of applying an effective amount of the anti-oxidant composition of claim 1 to said area of skin up to five days prior to said skin being subjected to EMR.

13. The method of claim 11, wherein said EMR is selected from the group consisting of sun exposure, laser exposure and radiation treatment.
